(19) United States
(12) Patent Application Publication
(10) Pub. No.: US 2025/0170330 A1
(43) Pub. Date: May 29, 2025

(54) ADAPTIVE DELIVERY PROFILES FOR AUTOMATED INSULIN DELIVERY TREATMENT SYSTEM

(71) Applicant: Tandem Diabetes Care, Inc, San Diego, CA (US)

(72) Inventor: Christian Merz, San Diego, CA (US)

(21) Appl. No.: 18/962,169

(22) Filed: Nov. 27, 2024

Related U.S. Application Data
(60) Provisional application No. 63/602,985, filed on Nov. 27, 2023.

(51) Int. Cl. A61M 5/172; A61B 5/00; A61B 5/145

(52) U.S. Cl. CPC A61M 5/1723; A61B 5/14532; A61B 5/4836

(57) ABSTRACT

Disclosed herein are systems and methods for closed loop and/or partial and/or hybrid closed loop therapy that can provide adaptive personal profiles for when glucose levels are not available and/or unreliable from a continuous glucose monitor (CGM), such as, for example, during a warmup period of the CGM sensor. The profile can include temporary delivery profile such as a CGM warmup profile that is activated specifically for the particular scenario and is designed to minimize the risk of blood glucose fluctuations until the glucose levels are available and reliable, such as when the CGM sensor is warmup up and closed loop therapy based on the sensor can be activated.

### PRIORITY CLAIM

This application claims priority to U.S. Provisional Application No. 63/602,985, filed Nov. 27, 2023, which is hereby incorporated herein by reference in its entirety.

### TECHNICAL FIELD

The present disclosure is directed generally to systems and methods relating to portable infusion pumps and more particularly to continuous glucose monitoring in infusion pump systems.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Some ambulatory infusion pumps are configured to receive glucose level data of the user from a continuous glucose monitoring (CGM) device that obtains measurements relating to glucose levels in the body. The pump can then use the glucose data in making therapy determinations. Some systems due so in a “closed loop” manner, in which medicament delivery is automatically adjusted based on the glucose levels. Conventionally, infusion pump systems have been limited by the ability of these sensors. For example, a CGM sensor generally has a “warmup period,” between insertion of the CGM sensor into interstitial tissue of a user and active use of the CGM sensor, where CGM data is not available or detected by the CGM sensor. During this period, users do not have access to CGM readings, and therefore the infusion pump is unable to provide delivery of insulin in conjunction with closed loop continuous glucose monitoring. Such systems therefore typically revert to a stored default basal profile during the warmup period.

### SUMMARY

Disclosed herein are systems and methods for closed loop and/or partial and/or hybrid closed loop therapy that can provide adaptive personal profiles for when glucose levels are not available and/or unreliable from a continuous glucose monitor (CGM), such as, for example, during a warmup period of the CGM sensor. The personal profile can include a temporary delivery profile such as a CGM warmup profile that is activated specifically for the particular scenario and is designed to minimize the risk of blood glucose fluctuations until the glucose levels are available and reliable, such as when the CGM sensor is warmup up and closed loop therapy based on the sensor can be activated.

In an embodiment, an ambulatory infusion pump system can include a pump mechanism configured to deliver insulin to a user, a communications interface configured to receive data indicative of glucose levels of a user detected by a continuous glucose monitoring sensor and at least one processor configured to receive the data indicative of glucose levels of the user and to determine therapy parameters for the user based on the glucose levels. The at least one processor can be configured to cause insulin to be delivered according to a closed loop mode that determines the therapy parameters for the user based on a default closed loop basal profile and varies a basal rate from the default closed loop basal profile based on the glucose levels to maintain the glucose levels within a closed loop target range. If the at least one processor determines the use of the glucose levels to deliver insulin according to the closed loop mode should be discontinued, it can activate a temporary basal profile configured to be used during a period when closed loop mode is temporarily discontinued and cause insulin to be delivered according to the temporary basal profile. The closed loop mode can be reactivated when it is determined that the glucose levels can be used to deliver insulin according to the closed loop mode.

In an embodiment, an ambulatory infusion pump system can include a pump mechanism configured to deliver insulin to a user, a communications interface configured to receive data indicative of glucose levels of a user detected by a continuous glucose monitoring sensor and at least one processor configured to receive the data indicative of glucose levels of the user and to determine therapy parameters for the user based on the glucose levels. The at least one processor can be configured to cause insulin to be delivered according to a closed loop mode that determines the therapy parameters for the user based on a default closed loop basal profile and varies a basal rate from the default closed loop basal profile based on the glucose levels to maintain the glucose levels within a closed loop target range. If the at least one processor detects that the CGM sensor has expired, it can exit the closed loop mode. If it is determined that a new CGM sensor has been inserted the at least one processor can cause insulin to be delivered according to a CGM warmup profile during a warmup period in which glucose levels from the CGM sensor are not available. The closed loop mode can be activated following completion of the warmup period.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 depicts an embodiment of a pump system according to the disclosure.

FIG. 2 depicts a block diagram representing an embodiment of a pump system according to the disclosure.

FIGS. 3A-3B depicts an embodiment of a pump system according to the disclosure.

FIG. 4 depicts a schematic representation of a pump system according to an embodiment of the disclosure.

FIG. 5 depicts a flowchart of methods steps for delivering medicament to a patient for treatment of diabetes according to the disclosure.

FIG. 6 depicts a flowchart of methods steps for delivering medicament to a patient for treatment of diabetes according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

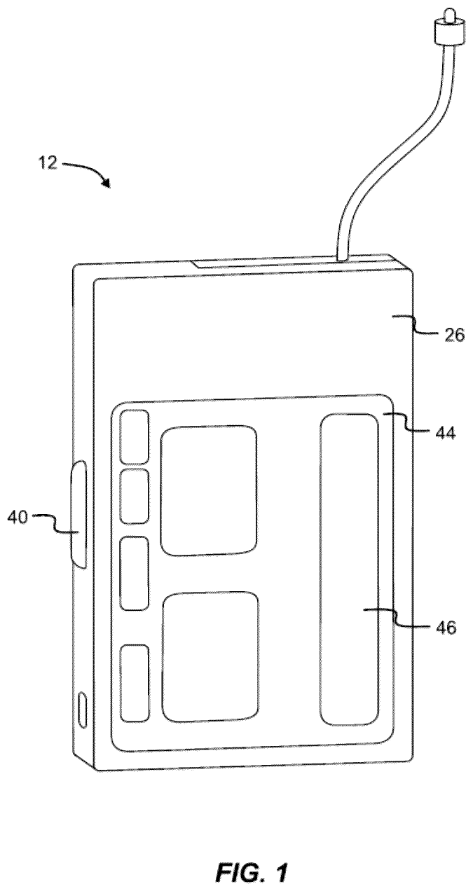

FIG. 1 depicts an exemplary medical device that can be used with embodiments of the disclosure. In this embodiment, the medical device is configured as a pump 12, such as an infusion pump, that can include a pumping or delivery mechanism and reservoir for delivering medicament to a patient and an output/display 44. The type of output/display 44 may vary as may be useful for a particular application (including no output/display with the device being remotely controlled). The output/display 44 may include an interactive and/or touch sensitive screen 46 having an input device such as, for example, a touch screen comprising a capacitive screen or a resistive screen. The pump 12 may additionally include a keyboard, microphone, or other input device known in the art for data entry, which may be separate from the display. The pump 12 may also include a capability to operatively couple to one or more blood glucose meters (BGMs) or continuous blood glucose monitors (CGMs) and/or one or more secondary devices such as a remote display, a remote control device, a laptop computer, personal computer, tablet computer, a mobile communication device such as a smartphone, a wearable electronic watch, smart ring, electronic health or fitness monitor, or personal digital assistant (PDA), a CGM display etc.

In one embodiment, the medical device can be a portable pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

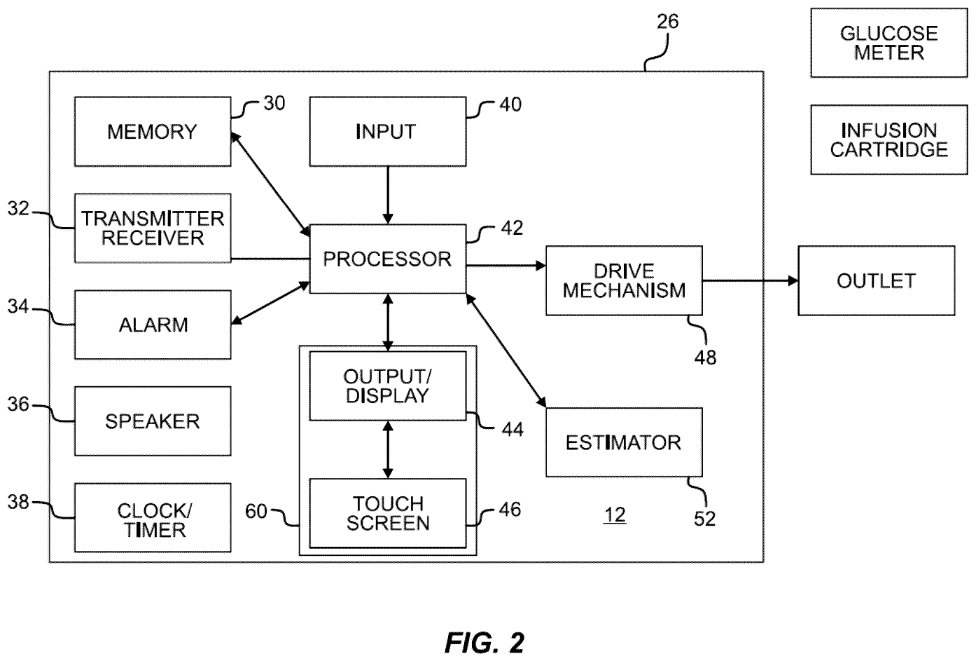

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor to receive signals and/or other data from one or more input devices, such as sensors that may sense pressure, temperature and/or other parameters.

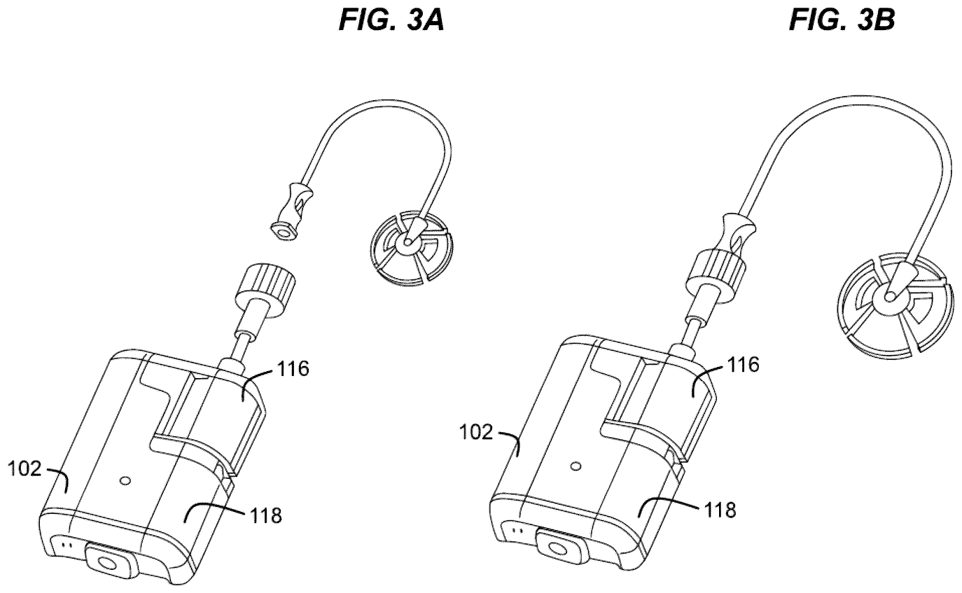

FIGS. 3A-3B depict a second infusion pump that can be used in conjunction with one or more embodiments of the ambulatory infusion pump system of the present disclosure. Pump 102 includes a pump drive unit 118 and a medicament cartridge 116. Pump 102 includes a processor that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, smart ring, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receives commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

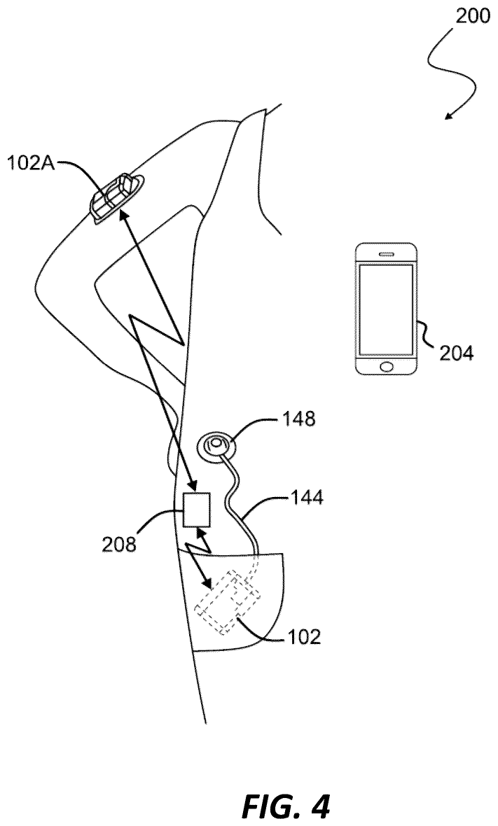

FIG. 4 depicts a schematic representation of a pump system 200 according to an embodiment. System 200 includes a user-wearable infusion pump such as pump 12 or pump 102 described above. In embodiments, a user can alternatively wear the pump 102A directly on the body or place the pump 102B in the user's pocket or other location near the body with infusion tubing 144 extending to an infusion set 148 on the user's body. The system 200 also includes a continuous glucose monitoring (CGM) sensor with a corresponding transmitter 208. The CGM sensor obtains measurements relating to glucose levels in the body and the transmitter communicates that information to the pump 102A/B. The pump 208 can then use the glucose data in making therapy determinations. The system can also include a one or more devices such as a smartphone 204 or other multi-purpose consumer electronic device capable of operating a software application to communicate with and/or control the pump and, alternatively or additionally, a dedicated remote control device designed specifically for use with pump 102A/102B. The smartphone 204 or other remote control device can in some embodiments also be capable of communication with CGM sensor/transmitter 208. In addition, the pump 102A/B and/or smartphone 204 or other remote control device can optionally communicate with additional devices such as, for example, a blood glucose meter or other analyte sensing device, an activity or other health monitor, etc.

With the infusion pump and CGM interfaced, the CGM can automatically transmit the CGM data to the pump and/or remote device. The pump can then use this data to automatically determine therapy parameters and suggest a therapy adjustment to the user or automatically deliver the therapy adjustment to the user. These therapy parameters including thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection).

For example, if the CGM readings indicate that the user has or is predicted to have a high blood glucose level, the ambulatory infusion system can automatically calculate an insulin dose sufficient to reduce the user's blood glucose level below a threshold level or to a target level and automatically deliver the dose. Alternatively, the ambulatory infusion system can automatically suggest a change in therapy upon receiving the CGM readings such as an increased insulin basal rate or delivery of a bolus but can require the user to accept the suggested change prior to delivery rather than automatically delivering the therapy adjustments.

By way of further example, if the CGM readings indicate that the user has or is predicted to have a low blood glucose level (hypoglycemia), the ambulatory infusion system can, for example, automatically reduce or suspend a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the patient address the hypoglycemic condition as necessary (e.g., ingest carbohydrates), singly or in any desired combination or sequence.

In some systems, the closed loop algorithm delivers insulin to the user according to the stored basal profile when the user's glucose levels remain within a target range. The general concept behind closed loop algorithms such as the algorithm described above is to use continuous glucose feedback to adjust insulin delivery when the stored baseline basal profile is delivering too much or not enough insulin. Because of the ability of the system while in closed loop mode to continually adjust delivery to maintain glucose at a desired level, many users set aggressive basal profiles attempting to maintain the user's glucose level in a narrower and lower range. Closed loop therapy as disclosed herein can include fully closed loop systems in which all insulin delivery is automatically determined based on glucose levels as well as partial or hybrid closed loop systems in which some insulin delivery is automatically determined based on glucose levels and other insulin deliveries or therapy parameters are carried out based on user input. For example, some systems may deliver basal insulin automatically based on glucose levels in a closed loop manner while receiving meal information from a user for delivery of meal boluses.

During an active CGM session, the CGM sensor will continually measure glucose levels of the user for use in therapy calculations in the system. However, CGM sessions are only activated after a period of time after a CGM sensor is inserted into the body of the user, which can be referred to as a “warm up” period. This warmup period (the period between insertion of a CGM sensor into interstitial tissue of the user and activation of a CGM session) is necessary because CGM sensors comprise porous material which requires wetting with interstitial tissue in order to absorb glucose and accurately monitor glucose levels. CGM readings are therefore unavailable to monitor the glucose levels of the user during this warmup period. The warmup period for CGM sensors can typically range anywhere from 30 minutes to 2 hours. As such, during the warmup period, the system is unable to provide automated delivery of insulin in conjunction with closed loop continuous glucose monitoring, as the system is also unable to access CGM data before activation of a CGM session.

During the CGM warmup period, automated insulin delivery systems typically revert to the user's stored basal profile and deliver insulin according to the stored profile independent of the user's glucose levels due to the unavailability and/or unreliability of that information from the CGM. However, as noted above many users set aggressive basal profiles attempting to maintain the user's glucose level in a narrower and lower range during closed loop therapy. During CGM warmup when glucose levels from the CGM sensor are not available and/or unreliable, such adjustments cannot be reliably made and delivering according to an aggressive stored profile can lead to undesirable low and high fluctuations in blood glucose.

In light of the above, systems and methods disclosed herein provide adaptive profiles in which a user can maintain a separate, stored basal profile that is applied during CGM warmup periods when glucose levels are not available from the CGM. To avoid potential undesirable fluctuations in glucose, this “warmup profile” can be configured to maintain the user's glucose levels within a wider and higher range than the stored profile used as the baseline during closed loop delivery. Reverting to this profile is different from reverting to a default open loop basal profile because it is designed specific for CGM warmup periods when CGM based closed loop therapy will begin in the near future, rather than being used any time the pump is not in a closed loop mode. In addition, in some embodiments the system may use the CGM values measured during the warmup period to automatically adjust delivery, with the more conservative CGM warmup profile providing a safeguard against the unreliability of the CGM data during this period. The system can then revert to the stored basal profile for closed loop therapy once standard closed loop delivery is resumed following the CGM warmup period. In some embodiments, the system can automatically revert to the stored profile upon detecting that a CGM warmup period has been completed.

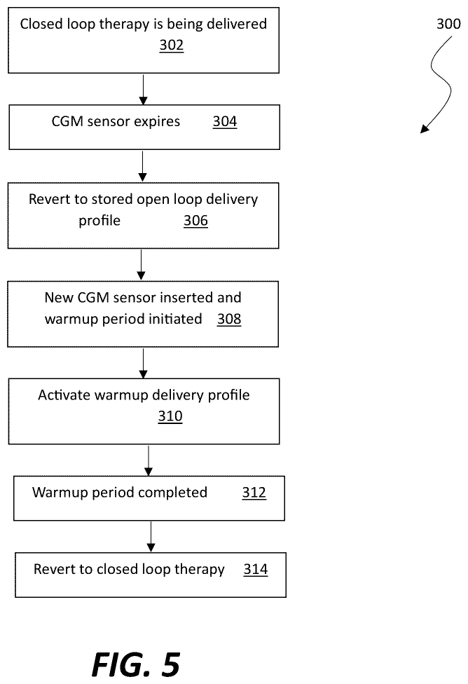

Referring now to FIG. 5, a flowchart of method steps for a method 300 of delivering medicament to a patient for treatment of diabetes according to the disclosure. At step 302, a medicament such as insulin is being delivered to a patient according to a closed loop algorithm that modifies a default basal profile of a user based on glucose levels of a user detected by a CGM sensor to maintain the user's glucose levels within a predefined range. When the CGM sensor expires at step 304, the system automatically reverts to open loop therapy at step 306. At step 308, a new CGM sensor is inserted to initiate a new CGM session, which will require the sensor to go through a warmup period in which glucose levels are not available and/or too unreliable for closed loop therapy. When the CGM session is initiated, rather than activating the default basal profile for closed loop, the system activates a warmup profile at step 310 designed for use during the CGM warmup period that is less aggressive than the default basal profile. In some embodiments the system delivers therapy directly based on the warmup profile, whereas in other embodiments the system may vary delivery based on glucose levels measured during the warmup period with the less aggressive warmup profile accounting for the unreliability of the CGM data during this period. At step 312 the warmup period is completed and at step 314 closed loop therapy is reactivated using the default basal profile. Although described above with the system reverting to the stored basal profile in open loop following sensor expiration and activating the warmup profile upon insertion of a new sensor, in some embodiments the system can activate the warmup profile upon expiration of a sensor and maintain the profile through the warmup period of a subsequently inserted sensor.

Although specifically described with respect to a CGM warmup period, it should be understood that the adaptive profile described above can be temporarily activated for any time period during with CGM readings are unavailable and/or unreliable. For example, CGM readings may also be unavailable if the sensor accidentally becomes disconnected from the user's body, the sensor battery dies or connectivity issues interrupt communications with the CGM. CGM readings could become unreliable due to various reasons, such as, for example, a CGM not having been calibrated within a required period of time. In any of these instances, a temporary delivery profile configured to maintain the user's glucose levels within a wider and higher range than the stored profile used as the baseline during closed loop delivery can be activated. Such CGM-unavailable temporary delivery profiles can be the same for any type of CGM unavailability/unreliability or can vary based on a specific reason that the temporary profile is needed. In embodiments where CGM data is being received but is considered unreliable, the system may deliver therapy solely based on the temporary profile or may continue to use the potentially unreliable data to automatically adjust therapy because of the wider and safer glucose range used by the temporary profile. Once the CGM data is available and/or reliable again, the system can automatically revert to the stored closed loop profile.

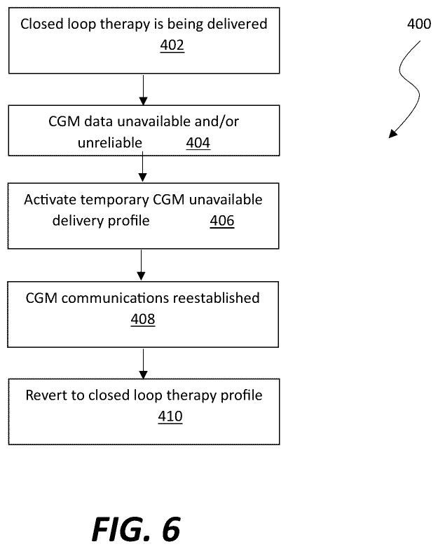

FIG. 6 depicts a flowchart of method steps for a method 400 of delivering medicament to a patient for treatment of diabetes according to the disclosure. At step 402, a medicament such as insulin is being delivered to a patient according to a closed loop algorithm that modifies a default basal profile of a user based on glucose levels of a user detected by a CGM sensor to maintain the user's glucose levels within a predefined range. If the CGM data becomes unavailable or unreliable at step 404, the system can activate a temporary CGM-unavailable delivery profile at step 406. This temporary delivery profile is configured to be safer than the default closed loop profile by being configured to maintain the user's glucose levels within a wider and higher range than the default profile when CGM readings are not being used or are considered unreliable. Once CGM communications are reestablished with available and reliable data at step 408, closed loop therapy is reactivated at step 410 using the default basal profile.

Adaptive personal profiles can also store additional profiles for other situations. For example, while some pumps include an “exercise mode” and a “sleep mode” that modify the default profile for exercise and sleep conditions, users according to the present disclosure can have various personalized profiles for these scenarios. For example, a user may have a different personal profile for high intensity exercise and low intensity exercise. These profiles may be user-selectable or the system may automatically select and/or modify an exercise profile based on feedback from an activity sensor or other device. Such profiles could include, for example, different basal rates, different glucose targets/ranges and either allow or disallow automatic correction boluses in closed loop therapy during different types of workouts. Users could further have many different profiles each for specific activities, such as separate personalized profiles for walking, running, biking, tennis, pickleball, etc.

In embodiments, an ambulatory infusion pump system, an ambulatory infusion pump system can include a pump mechanism configured to deliver insulin to a user, a communications interface configured to receive data indicative of glucose levels of a user detected by a continuous glucose monitoring sensor and at least one processor configured to receive the data indicative of glucose levels of the user and to determine therapy parameters for the user based on the glucose levels. The at least one processor can be configured to cause insulin to be delivered according to a closed loop mode that determines the therapy parameters for the user based on a default closed loop basal profile and varies a basal rate from the default closed loop basal profile based on the glucose levels to maintain the glucose levels within a closed loop target range. If the at least one processor determines the use of the glucose levels to deliver insulin according to the closed loop mode should be discontinued, it can activate a temporary basal profile configured to be used during a period when closed loop mode is temporarily discontinued and cause insulin to be delivered according to the temporary basal profile. The closed loop mode can be reactivated when it is determined that the glucose levels can be used to deliver insulin according to the closed loop mode.

In some embodiments, the temporary basal profile is configured to maintain the user's glucose levels within a wider target range than a target range of the default closed loop basal profile.

In some embodiments, a lowest glucose level and a highest glucose level in the wider target range are both higher than a lowest glucose level and a highest glucose level of the target range of the closed loop basal profile.

In some embodiments, the at least one processor is configured to determine the use of the glucose levels to deliver insulin according to the closed loop mode should be discontinued when the data indicative of glucose levels is not being received from the CGM sensor.

In some embodiments, the at least one processor is configured to determine that the glucose levels can be used to deliver insulin according to the closed loop mode when the data indicative of glucose levels are again being received from the CGM sensor.

In some embodiments, the at least one processor is configured to attempt to reestablish connectivity with the CGM sensor.

In some embodiments, the at least one processor is configured to determine the use of the glucose levels to deliver insulin according to the closed loop mode should be discontinued when the glucose levels are considered unreliable.

In some embodiments, the at least one processor is configured to consider the glucose levels to be unreliable during a warmup period for the CGM sensor.

In some embodiments, the at least one processor is configured automatically activate the closed loop mode upon an expiration of the warmup period.

In some embodiments, the at least one processor is configured to consider the glucose levels to be unreliable when the CGM sensor has not been calibrated within a threshold period of time.

In some embodiments, the at least one processor is configured automatically activate the closed loop mode upon calibration of the sensor.

In some embodiments, the at least one processor causes insulin to be delivered according to the temporary basal profile without accounting for the unreliable glucose levels.

In some embodiments, the at least one processor causes insulin to be delivered in varying amounts according to the temporary basal profile based on the unreliable glucose levels.

In embodiments, an ambulatory infusion pump system, an ambulatory infusion pump system can include a pump mechanism configured to deliver insulin to a user, a communications interface configured to receive data indicative of glucose levels of a user detected by a continuous glucose monitoring sensor and at least one processor configured to receive the data indicative of glucose levels of the user and to determine therapy parameters for the user based on the glucose levels. The at least one processor can be configured to cause insulin to be delivered according to a closed loop mode that determines the therapy parameters for the user based on a default closed loop basal profile and varies a basal rate from the default closed loop basal profile based on the glucose levels to maintain the glucose levels within a closed loop target range. If the at least one processor detects that the CGM sensor will be or is expiring it can exit the closed loop mode based on the expiration of the CGM sensor. If it is determined that a new CGM sensor has been inserted the at least one processor cause insulin to be delivered according to a CGM warmup profile during a warmup period of the new CGM sensor. The closed loop mode can be activated following completion of the warmup period.

In some embodiments, the CGM warmup profile is a modification of the default closed loop basal profile.

In some embodiments, the CGM warmup profile is configured to maintain the user's glucose levels within a wider target range than a target range of the default closed loop basal profile.

In some embodiments, a lowest glucose level and a highest glucose level in the wider target range are both higher than a lowest glucose level and a highest glucose level of the target range of the closed loop basal profile.

In some embodiments, the at least one processor causes insulin to be delivered according to the CGM warmup profile without accounting for glucose levels measured by the new CGM sensor during the warmup period.

In some embodiments, the at least one processor causes insulin to be delivered in varying amounts according to the CGM warmup profile based on the glucose levels measured by the new CGM sensor during the warmup period.

In some embodiments, the at least one processor is configured to cause insulin to be delivered according to a default open loop basal profile that is not varied based on glucose levels between when the CGM sensor expires and when the new CGM sensor is inserted.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708;; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924 and commonly owned U.S. patents applications Ser. Nos. 17/368,968; 17/896,492; 18/207,094; 18/398,543; 18/441,735; 18/474,839; 18/475,916; 18/478,552; 18/678,130; 18/700,168; 18/891,482; and 18/896,045.

For purposes of interpreting the claims, it is expressly intended that the provisions of 35 U.S.C. § 112(f) are not to be invoked unless the specific terms “means for” or “step for” are recited in a claim.

1. An ambulatory infusion pump system, comprising:
a pump mechanism configured to deliver insulin to a user;
a communications interface configured to receive data indicative of glucose levels of a user detected by a continuous glucose monitoring (CGM) sensor; and
at least one processor configured to receive the data indicative of glucose levels of the user and to determine therapy parameters for the user based on the glucose levels, the at least one processor configured to:
cause insulin to be delivered according to a closed loop mode that determines at least some therapy parameters for the user based on a default closed loop basal profile and varies a basal rate from the default closed loop basal profile based on the glucose levels to maintain the glucose levels within a closed loop target range;
determine that use of the glucose levels to deliver insulin according to the closed loop mode should be discontinued;
activate a temporary basal profile configured to be used during a period when closed loop mode is temporarily discontinued;
cause insulin to be delivered according to the temporary basal profile; and
reactivate the closed loop mode when it is determined that the glucose levels can be used to deliver insulin according to the closed loop mode.

2. The ambulatory infusion pump system of claim 1, wherein the temporary basal profile is configured to maintain the user's glucose levels within a wider target range than a target range of the default closed loop basal profile.

3. The ambulatory infusion pump system of claim 2, wherein a lowest glucose level and a highest glucose level in the wider target range are both higher than a lowest glucose level and a highest glucose level of the target range of the closed loop basal profile.

4. The ambulatory infusion pump system of claim 1, wherein the at least one processor is configured to determine the use of the glucose levels to deliver insulin according to the closed loop mode should be discontinued when the data indicative of glucose levels is not being received from the CGM sensor.

5. The ambulatory infusion pump system of claim 4, wherein the at least one processor is configured to determine that the glucose levels can be used to deliver insulin according to the closed loop mode when the data indicative of glucose levels are again being received from the CGM sensor.

6. The ambulatory infusion pump system of claim 4, wherein the at least one processor is configured to attempt to reestablish connectivity with the CGM sensor.

7. The ambulatory infusion pump system of claim 1, wherein the at least one processor is configured to determine the use of the glucose levels to deliver insulin according to the closed loop mode should be discontinued when the glucose levels are considered unreliable.

8. The ambulatory infusion pump system of claim 7, wherein the at least one processor is configured to consider the glucose levels to be unreliable during a warmup period for the CGM sensor.

9. The ambulatory infusion pump system of claim 8, wherein the at least one processor is configured automatically activate the closed loop mode upon an expiration of the warmup period.

10. The ambulatory infusion pump system of claim 7, wherein the at least one processor is configured to consider the glucose levels to be unreliable when the CGM sensor has not been calibrated within a threshold period of time.

11. The ambulatory infusion pump system of claim 10, wherein the at least one processor is configured automatically activate the closed loop mode upon calibration of the sensor.

12. The ambulatory infusion pump system of claim 7, wherein the at least one processor causes insulin to be delivered according to the temporary basal profile without accounting for the unreliable glucose levels.

13. The ambulatory infusion pump system of claim 7, wherein the at least one processor causes insulin to be delivered in varying amounts according to the temporary basal profile based on the unreliable glucose levels.

14. An ambulatory infusion pump system, comprising:
a pump mechanism configured to deliver insulin to a user;
a communications interface configured to receive data indicative of glucose levels of a user detected by a continuous glucose monitoring (CGM) sensor; and
at least one processor configured to receive the data indicative of glucose levels of the user and to determine therapy parameters for the user based on the glucose levels, the at least one processor configured to:
cause insulin to be delivered according to a closed loop mode that determines at least some therapy parameters for the user based on a default closed loop basal profile and varies a basal rate from the default closed loop basal profile based on the glucose levels to maintain the glucose levels within a closed loop target range;
detect that the CGM sensor will be or is expiring;
exit the closed loop mode based on the expiration of the CGM sensor;
determine that a new CGM sensor has been inserted;
cause insulin to be delivered according to a CGM warmup profile during a warmup period of the new CGM sensor; and
reactivate the closed loop mode following completion of the warmup period.

15. The ambulatory infusion pump system of claim 14, wherein the CGM warmup profile is a modification of the default closed loop basal profile.

16. The ambulatory infusion pump system of claim 14, where in the CGM warmup profile is configured to maintain the user's glucose levels within a wider target range than a target range of the default closed loop basal profile.

17. The ambulatory infusion pump system of claim 14, wherein a lowest glucose level and a highest glucose level in the wider target range are both higher than a lowest glucose level and a highest glucose level of the target range of the closed loop basal profile.

18. The ambulatory infusion pump system of claim 14, wherein the at least one processor causes insulin to be delivered according to the CGM warmup profile without accounting for glucose levels measured by the new CGM sensor during the warmup period.

19. The ambulatory infusion pump system of claim 14, wherein the at least one processor causes insulin to be delivered in varying amounts according to the CGM warmup profile based on the glucose levels measured by the new CGM sensor during the warmup period.

20. The ambulatory infusion pump system of claim 14, wherein the at least one processor is configured to cause insulin to be delivered according to a default open loop basal profile that is not varied based on glucose levels between when the CGM sensor expires and when the new CGM sensor is inserted.
